(19) United States
(12) Patent Application Publication
(10) Pub. No.: US 2014/0005498 A1
(43) Pub. Date: Jan. 2, 2014

(54) MEDICAL APPLICATION FOR MOBILE ELECTRONIC DEVICE

(71) Applicant: Russell Burry, Lafayette, IN (US)

(72) Inventor: Russell Burry, Lafayette, IN (US)

(21) Appl. No.: 13/932,159

(22) Filed: Jul. 1, 2013

Related U.S. Application Data
(60) Provisional application No. 61/666,633, filed on Jun. 29, 2012.

(51) Int. Cl. A61B 5/00

(52) U.S. Cl. CPC A61B 5/0004
USPC 600/301; 600/595

(57) ABSTRACT

A method can include a camera of a mobile electronic device capturing a photo of at least one eye of a patient, a photo of a finger of the patient, and a photo of at least one type of medication taken by the patient. The method can also include administering a motor test to the patient and storing in a database results of the motor test along with the captured photos.

### CROSS-REFERENCE TO RELATED APPLICATION

This application claims the benefit of U.S. Provisional Patent Application Ser. No. 61/666,633, titled “MEDICAL APPLICATION FOR MOBILE ELECTRONIC DEVICE” and filed on Jun. 29, 2012, the content of which is hereby incorporated by reference herein in its entirety.

### TECHNICAL FIELD

The disclosed technology relates generally to medical applications and, more particularly, to medical applications suitable for use in conjunction with mobile electronic devices such as smartphones and tablet computers.

### BACKGROUND

Mobile electronic devices, such as smartphones, tablet computers, and personal digital assistants (PDAs), have been used more and more for everyday activities as well as specialized events and interactions. However, current medical applications have not been adapted to take advantage of such mobile electronic device. Thus, a need remains for medical devices suitable for use in conjunction with mobile electronic devices such as smartphones and tablet computers.

### BRIEF DESCRIPTION OF THE DRAWINGS

FIG. 1 illustrates an example of a registration screen presented to a patient in accordance with certain embodiments of the disclosed technology.

FIG. 2 illustrates an example of a home screen in accordance with certain embodiments of the disclosed technology.

FIG. 3 illustrates a first step screen corresponding to a first step of an example of a test presented to the patient in accordance with certain embodiments of the disclosed technology.

FIG. 4 illustrates a second step screen corresponding to a second step of the example test presented to the patient in accordance with certain embodiments of the disclosed technology.

FIG. 5 illustrates a third step screen corresponding to a third step of the example test presented to the patient in accordance with certain embodiments of the disclosed technology.

FIG. 6 illustrates a fourth step screen corresponding to a fourth step of the example test presented to the patient in accordance with certain embodiments of the disclosed technology.

FIG. 7 illustrates a fifth step screen corresponding to a fifth step of the example test presented to the patient in accordance with certain embodiments of the disclosed technology.

FIG. 8 illustrates a sixth step screen corresponding to a sixth step of the example test presented to the patient in accordance with certain embodiments of the disclosed technology.

FIG. 9 illustrates an example of an exit screen presented to the patient in accordance with certain embodiments of the disclosed technology.

FIG. 10 illustrates an example of a settings screen presented to the patient in accordance with certain embodiments of the disclosed technology.

FIG. 11 is a flowchart illustrating an example of a computer-implemented method of collecting, sending, and synchronizing patient data in accordance with certain embodiments of the disclosed technology.

FIG. 12 illustrates an example of a first system in accordance with certain embodiments of the disclosed technology.

FIG. 13 is a flowchart illustrating an example of a computer-implemented method of interacting with various databases in accordance with certain embodiments of the disclosed technology.

FIG. 14 illustrates an example of a second system in accordance with certain embodiments of the disclosed technology.

### DETAILED DESCRIPTION

Embodiments of the disclosed technology may be based upon the requirements of a doctor to assist with the management of his or her patients who are taking specific drugs. The disclosed technology advantageously allows a doctor to diagnose a particular problem of his or her patient in situations where the patient is not physically present. In addition, the doctor can measure the accurate recovery of his or her patient with use of medicines. Further, the patient can take the test easily without even needing to visit his or her doctor in person.

Embodiments of the disclosed technology may be implemented using any of a number of programming languages, such as the Android Java Development Environment, for example, or any other suitable development environment and may be implemented using any of a number of mobile phones, such as Android mobile phones, for example, or any other suitable mobile electronic device. Certain implementations include Android-based mobile device having touchscreen capability and resolutions of 480×800 as well as a front-facing camera. Embodiments may be configured to run on the Apple iPhone, other mobile platforms, and home or laptop personal computers (PCs).

In certain embodiments, the first screen presented to a user is a welcome screen that presents to the patient a logo for the application and/or the company branding logo as well as a welcome message. This splash screen may be set to occur during every startup of the mobile device, for example, and may have a predetermined duration, e.g., 2-3 seconds.

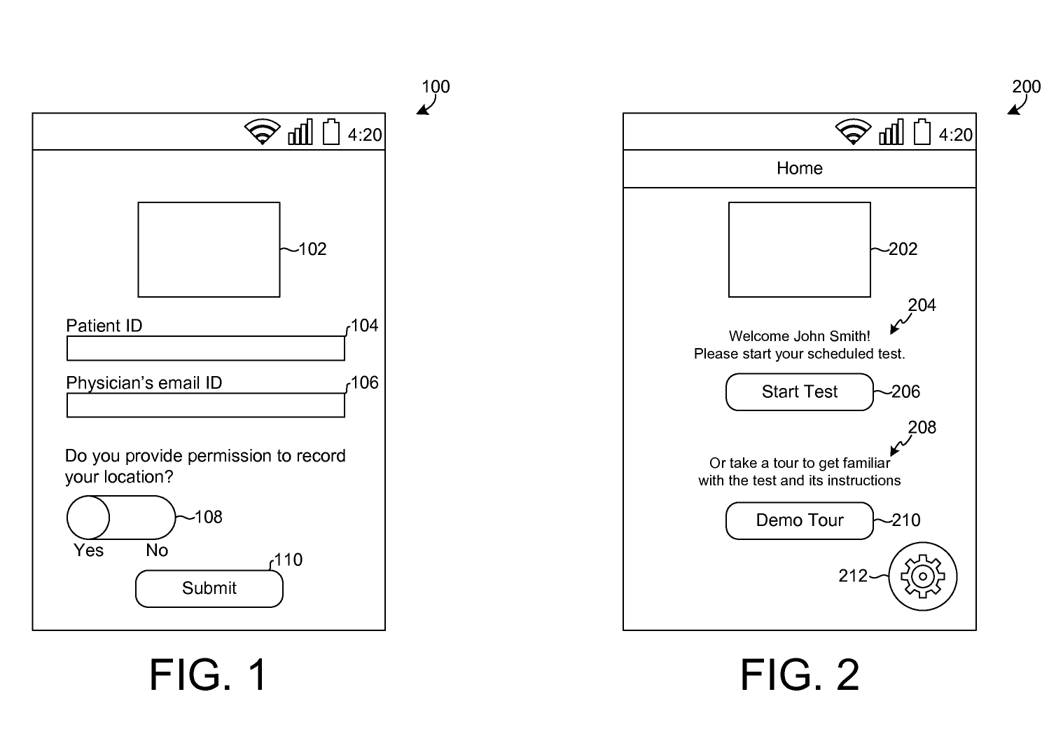

FIG. 1 illustrates an example of a registration screen 100 presented to a patient in accordance with certain embodiments of the disclosed technology. This registration screen 100 of the application may be set to appear only during the first startup of the mobile device, for example. The registration screen 100 may visually present a company logo or other image 102 to the patient. The registration screen 100 may prompt the patient to enter a patient identifier 104, e.g., Patient Id, and provider identifier 106, e.g., Physician's Email Id. Either or both of the patient identifier 104 and provider identifier 106 may be saved in a particular data store, e.g., a local database.

In certain embodiments, the registration screen 100 may also request a verification 108 from the patient as to whether he or she would permit the device to record his or her current location when the forthcoming test is taken by the patient. In situations where the patient permits the application to send out his or her current location after taking the test, such information may be included in a resulting communication, e.g., email message, that is sent to the patient's doctor.

Responsive to the patient clicking on a Submit button 110 on the registration screen 100, a home screen may be presented to the patient.

FIG. 2 illustrates an example of a home screen 200 in accordance with certain embodiments of the disclosed technology. The home screen 200 may visually present a company logo or other image 202 to the patient. Responsive to the patient selecting a first button 210, e.g. Demo Tour, on the home screen 200, the application may present to the patient a guide that explains how to use the application. Responsive to the patient selecting a second button 206, e.g., Start Test, on the home screen 200, the application may initiate a test. Messages 208 and 204 may be visually presented to the patient to provide a description associated with the corresponding first and second buttons 210 and 206, respectively.

Responsive to the patient clicking on a Setting button 212 on the home screen 200, a settings screen may be presented to the patient.

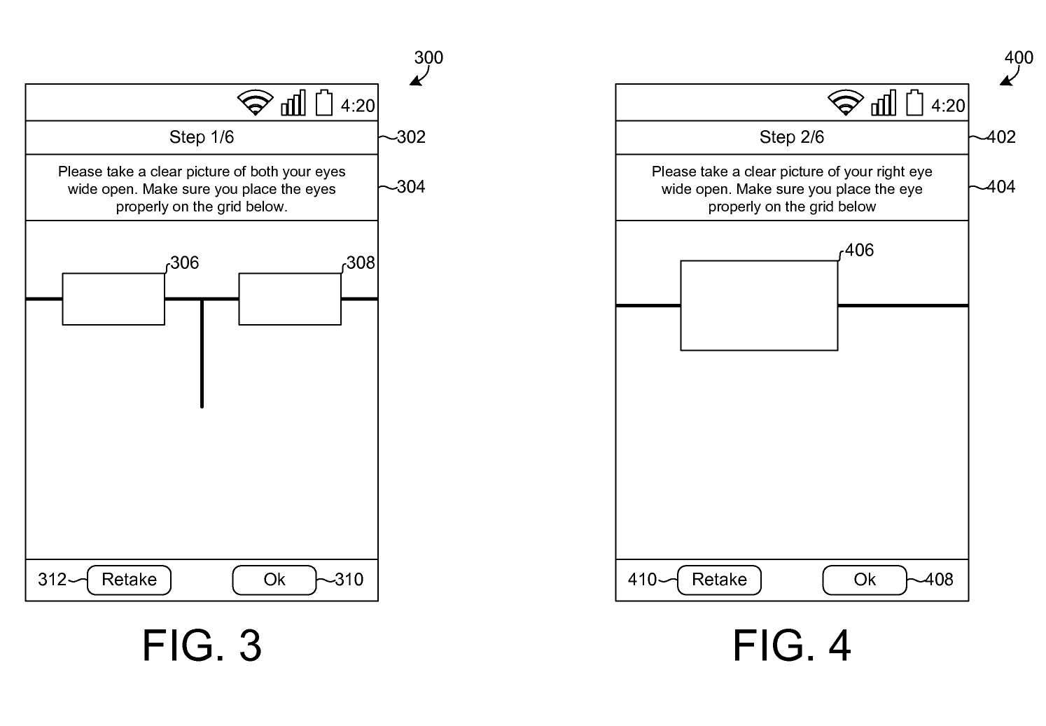

FIG. 3 illustrates a first step screen 300 corresponding to a first step of an example of a test presented to the patient in accordance with certain embodiments of the disclosed technology. A first section 302 of the first step screen 300 may visually communicate where the patient currently is with regard to the test. A second section 304 of the first step screen 300 may be visually presented to the patient to provide a description associated with the first step of the test.

A front camera of the patient's mobile device may start and a grid may be visually presented to the patient to guide the patient to ensure that the camera is positioned correctly. The grid may include first and second designated areas 306 and 308 that the patient may use to align his or her eyes with the front camera of the mobile device. For example, the patient may hold the mobile device and position it such that the patient can see each of his or her left and right eyes in one of the first and second areas 306 and 308, at which point the user may select a button or otherwise cause the front camera to capture a photograph of his or her eyes as presented by the first step screen 300 at the time of the capture.

If the user is satisfied that his or her left and right eyes are within the first and second areas 306 and 308 in the photo captured by the front camera, he or she may select an OK button 310, which causes the application to save the photo. If the user is not satisfied with the captured photo or wants to re-take the photo for some other reason, he or she may select a Retake button 312, which causes the application to restart the first step screen 300.

FIG. 4 illustrates a second step screen 400 corresponding to a second step of the example test presented to the patient in accordance with certain embodiments of the disclosed technology. A first section 402 of the second step screen 400 may visually communicate where the patient currently is with regard to the test. A second section 404 of the second step screen 400 may be visually presented to the patient to provide a description associated with the second step of the test.

The front camera of the patient's mobile device may start again and a grid may be visually presented to the patient to guide the patient to ensure that the camera is positioned correctly. The grid may include a designated area 406 that the patient may use to align his or her right eye with the front camera of the mobile device. For example, the patient may hold the mobile device and position it such that the patient can see his or her right eye in the designated area 406, at which point the user may select a button or otherwise cause the front camera to capture a photograph of his or her right eye as presented by the second step screen 400 at the time of the capture.

If the user is satisfied that his or her right eye is at least substantially within the designated area 406 in the photo captured by the front camera, he or she may select an OK button 408, which causes the application to save the photo. If the user is not satisfied with the captured photo or wants to re-take the photo for some other reason, he or she may select a Retake button 410, which causes the application to restart the second step screen 400.

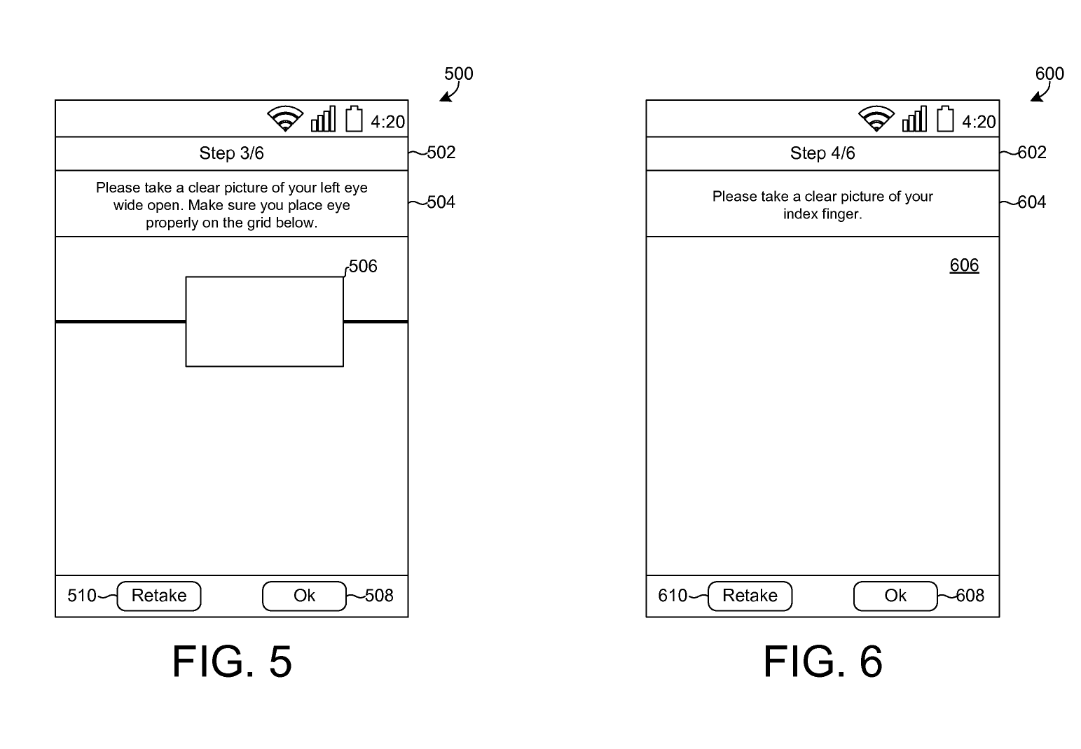

FIG. 5 illustrates a third step screen 500 corresponding to a third step of the example test presented to the patient in accordance with certain embodiments of the disclosed technology. A first section 502 of the third step screen 500 may visually communicate where the patient currently is with regard to the test. A second section 504 of the third step screen 500 may be visually presented to the patient to provide a description associated with the third step of the test.

The front camera of the patient's mobile device may start again and a grid may be visually presented to the patient to guide the patient to ensure that the camera is positioned correctly. The grid may include a designated area 506 that the patient may use to align his or her left eye with the front camera of the mobile device. For example, the patient may hold the mobile device and position it such that the patient can see his or her left eye in the designated area 506, at which point the user may select a button or otherwise cause the front camera to capture a photograph of his or her left eye as presented by the third step screen 500 at the time of the capture.

If the user is satisfied that his or her left eye is at least substantially within the designated area 506 in the photo captured by the front camera, he or she may select an OK button 508, which causes the application to save the photo. If the user is not satisfied with the captured photo or wants to re-take the photo for some other reason, he or she may select a Retake button 510, which causes the application to restart the third step screen 500.

FIG. 6 illustrates a fourth step screen 600 corresponding to a fourth step of the example test presented to the patient in accordance with certain embodiments of the disclosed technology. A first section 602 of the fourth step screen 600 may visually communicate where the patient currently is with regard to the test. A second section 604 of the fourth step screen 600 may be visually presented to the patient to provide a description associated with the fourth step of the test.

The front camera of the patient's mobile device may start again and a grid may be visually presented to the patient to guide the patient to ensure that the camera is positioned correctly. The grid may include a designated area 606 that the patient may use to align his or her index finger with the front camera of the mobile device. For example, the patient may hold the mobile device and position it such that the patient can see his or her index finger in the designated area 606, at which point the user may select a button or otherwise cause the front camera to capture a photograph of his or her index finger as presented by the fourth step screen 600 at the time of the capture.

If the user is satisfied that his or her index finger is at least substantially within the designated area 606 in the photo captured by the front camera, he or she may select an OK button 608, which causes the application to save the photo. If the user is not satisfied with the captured photo or wants to re-take the photo for some other reason, he or she may select a Retake button 610, which causes the application to restart the fourth step screen 600.

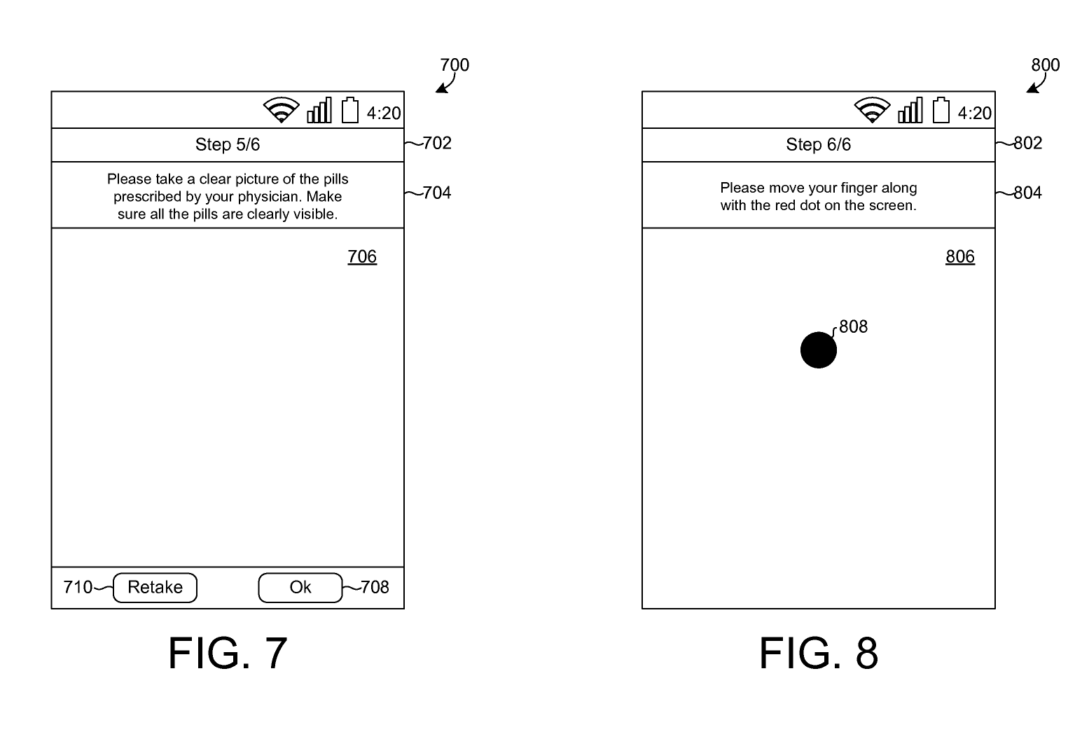

FIG. 7 illustrates a fifth step screen 700 corresponding to a fifth step of the example test presented to the patient in accordance with certain embodiments of the disclosed technology. A first section 702 of the fifth step screen 700 may visually communicate where the patient currently is with regard to the test. A second section 704 of the fifth step screen 700 may be visually presented to the patient to provide a description associated with the fifth step of the test.

A front or back camera of the patient's mobile device may start and a grid may be visually presented to the patient to guide the patient to ensure that the camera is positioned correctly. The grid may include a designated area 706 that the patient may use to align his or her pills, pill bag, or other pill container (referred to collectively herein as “pills”) with the camera of the mobile device. For example, the patient may hold the mobile device and position it such that the patient can see his or her pills container in the designated area 706, at which point the user may select a button or otherwise cause the camera to capture a photograph of his or her pills as presented by the fifth step screen 700 at the time of the capture.

If the user is satisfied that his or her pills are at least substantially within the designated area 706 in the photo captured by the front camera, he or she may select an OK button 708, which causes the application to save the photo. If the user is not satisfied with the captured photo or wants to re-take the photo for some other reason, he or she may select a Retake button 710, which causes the application to restart the fifth step screen 700.

FIG. 8 illustrates a sixth step screen 800 corresponding to a sixth step of the example test presented to the patient in accordance with certain embodiments of the disclosed technology. A first section 802 of the sixth step screen 800 may visually communicate where the patient currently is with regard to the test. A second section 804 of the sixth step screen 800 may be visually presented to the patient to provide a description associated with the sixth step of the test.

The sixth step may include the administering of a motor test. This may be done by visually presenting within a third section 806 an object 808, e.g., a red dot, to the patient. The patient may be directed to move his or her fingertip, e.g., on a touchpad or other touch surface of the mobile device, along with the object 808. Once the patient has moved his or her finger along with the object 808 as directed, the test may be complete and the application may advance to an exit screen.

It should be noted that, during the course of a test such as the example test illustrated and described above with regard to FIGS. 3-8, the instructions for any or each step may be presented to the patient in a visible manner, e.g., textually, and/or presented to the patient in an audible manner, e.g., by way of a text-to-voice feature that allows the mobile device to “speak” the instructions to the patient.

Once all of the steps of the test, e.g., first through sixth steps, have been completed, a report of the completed test, including the resulting information and/or results, may be sent to the patient's doctor, e.g., by way of an email message sent to the email address of the doctor, e.g., the Physician's Email Id entered at the registration screen, along with the Patient Id.

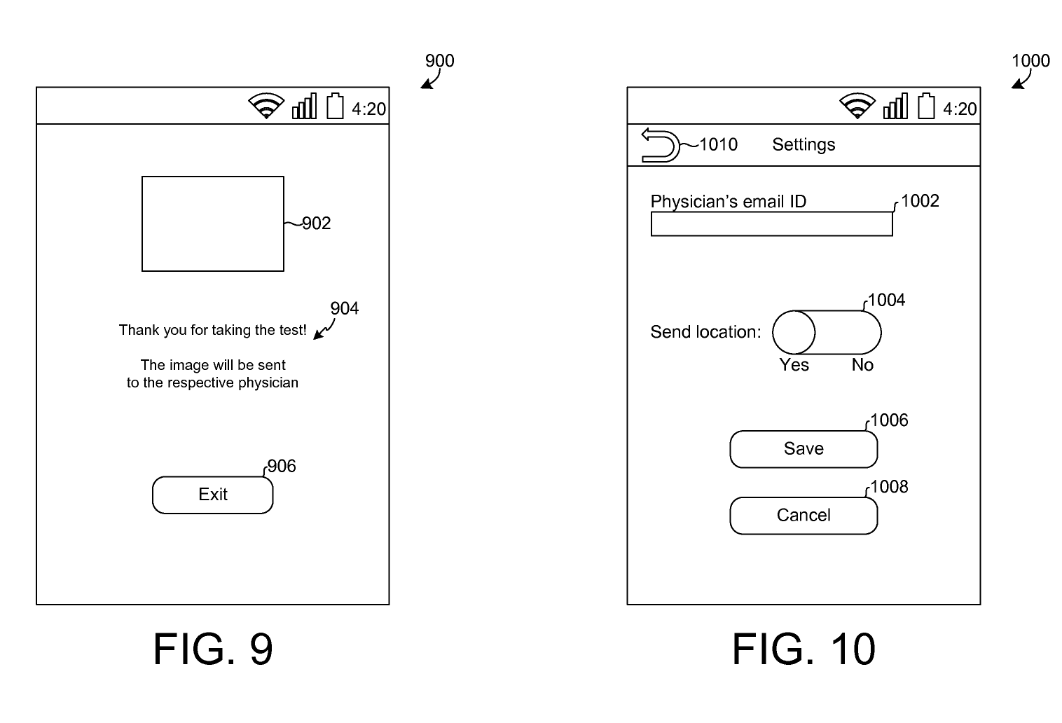

FIG. 9 illustrates an example of an exit screen 900 presented to the patient in accordance with certain embodiments of the disclosed technology. The exit screen 900 may visually present a company logo or other image 902 to the patient. Responsive to the patient selecting an Exit button 906 on the exit screen 900, the application may close. A message 904 may be visually presented to the patient to provide an indication that the test is complete, that certain information from the test will be sent to the patient's provider, or both.

In certain embodiments, the application may send the captured screen data and motor test results to servers for storage, back office processing, or both. The information may be stored locally, e.g., by the mobile electronic device, remotely, e.g., at a remote server or database, or both. The back office processing may include matching the patient's captured test material and results, such as the images and motor test results, to the base data that was captured during a provider set up process. A provider, or the application itself, may establish a delta variance from the base data, or other prior data, to incremental data. In certain embodiments, a provider may be notified in the event of an identified delta occurrence.

In certain embodiments, the provider set up process may include a capture of the original patient data, such as the images of the patient's left eye and/or right eye, home location, finger prints, voice print, IRIS imprint, and results of the motor test. This captured data may be concurrently or subsequently stored on servers for concurrent or subsequent comparison to the patient's intermittent test results.

In certain embodiments, one or more servers may initiate proactive communication with the patient. For example, the server(s) may initiate one or more calls on a random basis between set hours of the day as determined by the doctor. The server(s) may also send the patient an email message that he or she is or will soon be due to take the test. The application may allow the patient a few minutes to begin the test. If the patient fails to start the test within the established time frame, such information may be sent to the server(s) and subsequently sent to the doctor.

Implementations of the disclosed technology may include one or more security measures to ensure that the person taking the test on the mobile electronic device is indeed the patient. For example, the application may use any or all of voice imprint matching, IRIS recognition, finger print matching, right eye recognition, and left eye recognition to determine that the person taking the test is the patient.

FIG. 10 illustrates an example of a settings screen 1000 presented to the patient in accordance with certain embodiments of the disclosed technology. This settings screen 1000 of the application may allow the patient to verify or edit a provider identifier 1002, e.g., Physician's Email Id. In certain embodiments, the settings screen 1002 may also request a verification 1004 from the patient as to whether he or she would permit the device to include the patient's location, e.g., in a reporting message or otherwise in connection with results of the patient's test.

Responsive to the patient clicking on a Save button 1006 on the settings screen 1000, the changes (if any) may be saved. Responsive to the patient clicking on a Cancel button 1008 on the settings screen 1000, the application may close.

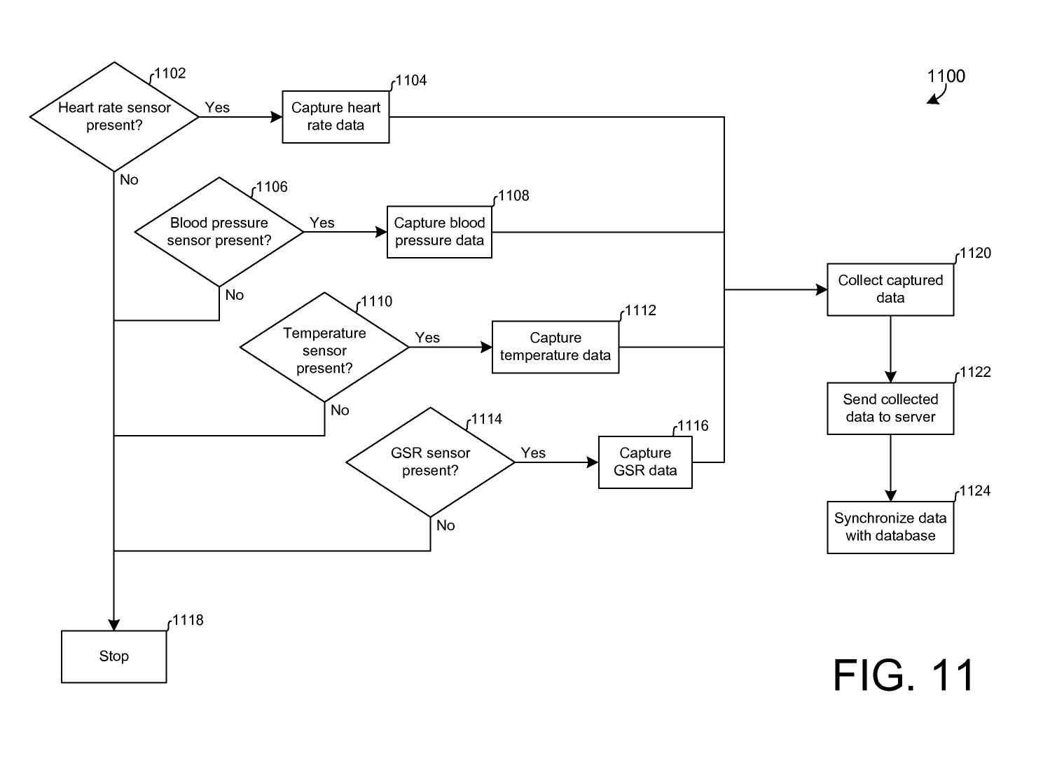

FIG. 11 is a flowchart illustrating an example of a computer-implemented method 1100 of collecting, sending, and synchronizing patient data in accordance with certain embodiments of the disclosed technology.

At 1102, a determination is made as to whether a heart rate sensor is present, e.g., coupled with the patient. If so, heart rate data of the patient may be captured as indicated at 1104; otherwise, any processing with regard to heart rate data of the patient is stopped as indicated at 1118. It should be noted that, in embodiments where the heart rate sensor is present, the heart rate data of the patient may be collected while the patient is taking a test such as the example test described above and illustrated by FIGS. 3-8. Such data capture may commence as the patient begins the test, for example.

At 1106, a determination is made as to whether a blood pressure sensor is present, e.g., coupled with the patient. If so, blood pressure data of the patient may be captured as indicated at 1108; otherwise, any processing with regard to blood pressure data of the patient is stopped as indicated at 1118. It should be noted that, in embodiments where the blood pressure sensor is present, the blood pressure data of the patient may be collected while the patient is taking a test such as the example test described above and illustrated by FIGS. 3-8. Such data capture may commence as the patient begins the test, for example.

At 1110, a determination is made as to whether a temperature sensor is present, e.g., coupled with the patient. If so, temperature data of the patient may be captured as indicated at 1112; otherwise, any processing with regard to temperature data of the patient is stopped as indicated at 1118. It should be noted that, in embodiments where the temperature sensor is present, the temperature data of the patient may be collected while the patient is taking a test such as the example test described above and illustrated by FIGS. 3-8. Such data capture may commence as the patient begins the test, for example.

At 1114, a determination is made as to whether a Galvanic Skin Response (GSR) sensor is present, e.g., coupled with the patient. If so, GSR data of the patient may be captured as indicated at 1116; otherwise, any processing with regard to GSR data of the patient is stopped as indicated at 1118. It should be noted that, in embodiments where the GSR sensor is present, the GSR data of the patient may be collected while the patient is taking a test such as the example test described above and illustrated by FIGS. 3-8. Such data capture may commence as the patient begins the test, for example.

At 1120, any patient data captured at any or all of 1104, 1108, 1112, and 1116 may be collected. As the patient data may be collected by any or all of 1104, 1108, 1112, and 1116 at least partially concurrently or separately, so may the collecting of such at 1120 be performed at least partially concurrently or separately. Such collected data may be sent to a server as indicated at 1122. The collected patient data may be sent in connection with a test such as the example test described above and illustrated by FIGS. 3-8. In certain embodiments, the patient data may be streamed to the server, e.g., every five seconds. The patient data may also be synchronized with a database, as indicated at 1124.

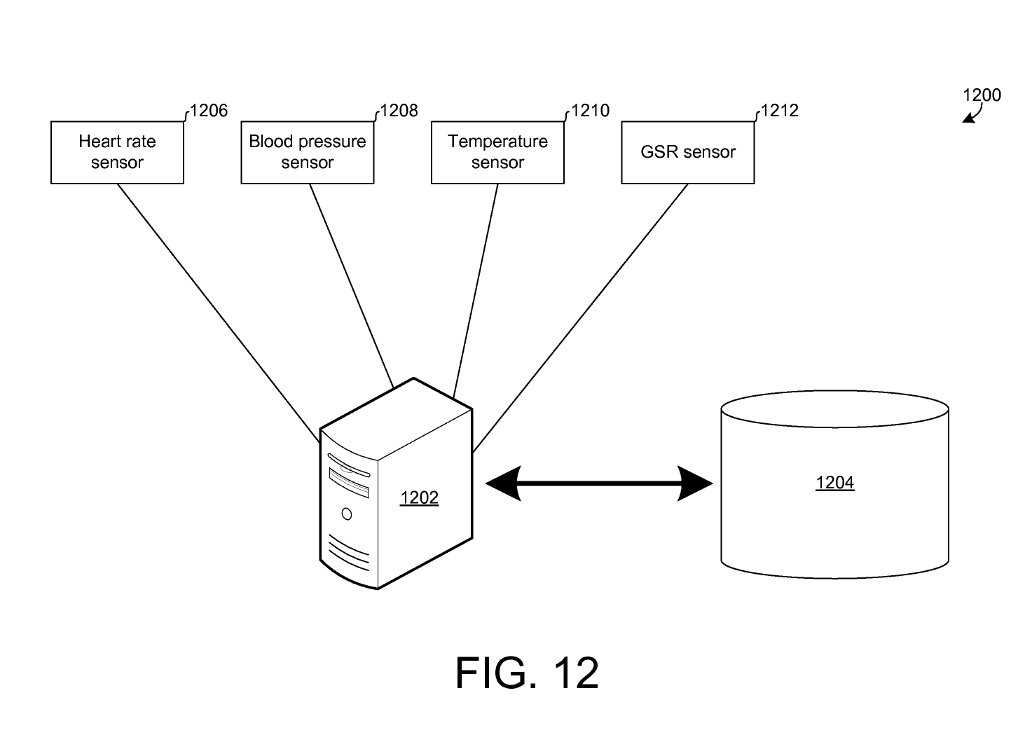

FIG. 12 illustrates an example of a first system 1200 in accordance with certain embodiments of the disclosed technology. In the example, an application running on a device 1202 may interact with any or all of various types of sensors such as a heart rate sensor 1206, a blood pressure sensor 1208, a temperature sensor 1210, and a GSR sensor 1212. In certain embodiments where one of the sensors 1206, 1208, 1210, 1212 is capturing patient data, the sensor, application, or both may shut down responsive to a determination that the corresponding signal has not been received from the patient for a specified period of time, e.g., thirty minutes.

The device 1202 may be a mobile electronic device such as a smartphone or tablet computer, for example. The application running on the device 1202 may also interact with a database 1204, e.g., to store or otherwise synchronize patient data therewith.

It will be appreciated that there are various other types of patient data that may be captured, collected, synchronized, or otherwise processed that are not illustrated by FIGS. 11 and 12. Such types of patient data may include, but are not limited to, blood sugar level data and brain EEG data.

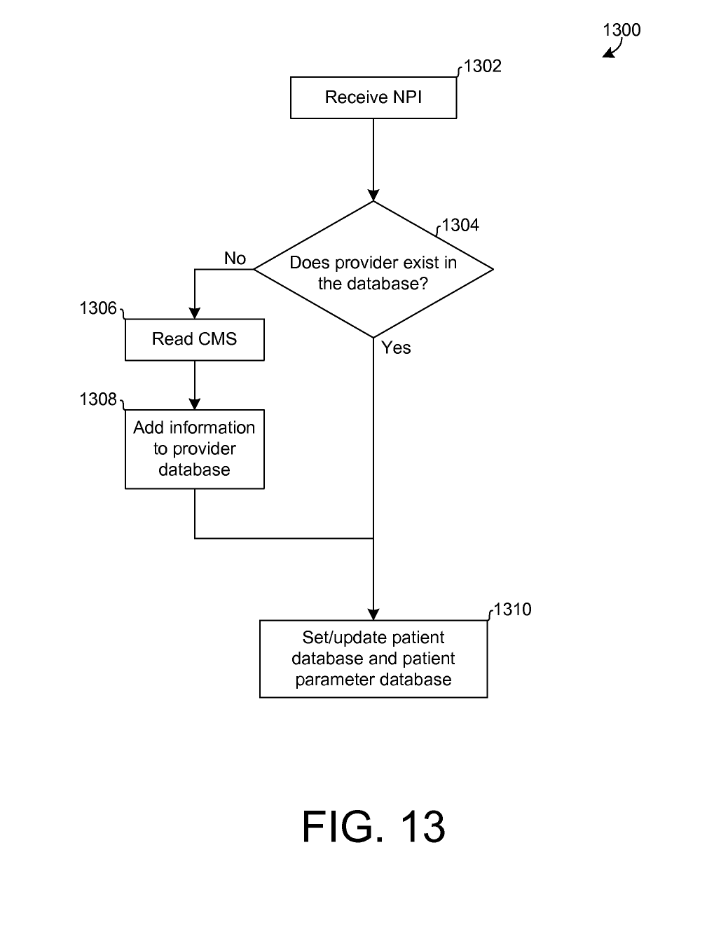

FIG. 13 is a flowchart illustrating an example of a computer-implemented method 1300 of interacting with various databases in accordance with certain embodiments of the disclosed technology. At 1302, a national provider identifier (NPI) is received. A determination is made at 1304 as to whether a record for a provider associated with the NPI exists in a provider database. If so, either or both of a patient database and a patient parameter database may be set or updated as indicated at 1310; otherwise, a Centers for Medicare and Medicaid Services (CMS) system or database may be accessed or otherwise read, as indicated at 1306, and a provider database may then be updated accordingly as indicated at 1308.

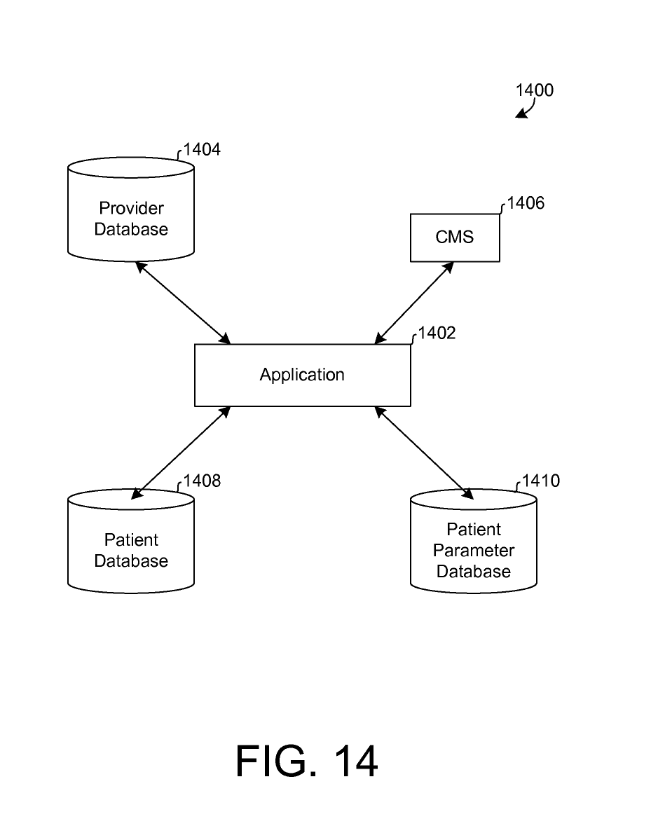

FIG. 14 illustrates an example of a second system 1400 in accordance with certain embodiments of the disclosed technology. In the example, an application 1402, e.g., running on a mobile electronic device, may interact with any or all of a provider database 1404, a CMS system or database 1406, a patient database 1408, and a patient parameter database 1410.

In certain embodiments, the provider database 1404 may include a record for each of a number of providers. Each record may include any or all of the following types of information: provider practice name, provider display name, state license identification, state license begin date, state license end date, date setup, date modified, and contact information such as email identification, first name, last name, primary number, secondary number(s), fax number, setup date, and end date.

In certain embodiments, either or both of the patient database 1408 and the patient parameter database 1410 may include a record for each of a number of patients. Each record may include any or all of the following types of information: NPI, sequence number, test parameter sequence number, parameter start date, home GPS location, PDA device type, PDA contact number, contact email, contact preference, contact preference order, contact failure process indicator, contact standard email message, and contact standard voice message. Each record may also include information pertaining to the patient's eye(s), finger(s), pills, other information pertaining to the patient's interactions with the application during a test, or any combination thereof. In certain embodiments, the NPI, patient sequence number, date, time, or a combination thereof may be used to identify each record in the database.

Having described and illustrated the principles of the invention with reference to illustrated embodiments, it will be recognized that the illustrated embodiments may be modified in arrangement and detail without departing from such principles, and may be combined in any desired manner. And although the foregoing discussion has focused on particular embodiments, other configurations are contemplated. In particular, even though expressions such as “according to an embodiment of the invention” or the like are used herein, these phrases are meant to generally reference embodiment possibilities, and are not intended to limit the invention to particular embodiment configurations. As used herein, these terms may reference the same or different embodiments that are combinable into other embodiments.

Consequently, in view of the wide variety of permutations to the embodiments described herein, this detailed description and accompanying material is intended to be illustrative only, and should not be taken as limiting the scope of the invention. What is claimed as the invention, therefore, is all such modifications as may come within the scope and spirit of the following claims and equivalents thereto.

1. A method, comprising:
a camera of a mobile electronic device capturing a photo of at least one eye of a patient;
the camera capturing a photo of a finger of the patient;
the camera capturing a photo of at least one type of medication taken by the patient;
the mobile electronic device administering a motor test to the patient; and
a database storing the photo of the at least one eye, the photo of the finger, the photo of the medication, and results of the motor test.

2. The method of claim 1, further comprising the camera capturing a photo of both eyes of the patient.

3. The method of claim 1, wherein administering the motor test comprises visually presenting to the patient a dot on a touchscreen of the mobile electronic device.

4. The method of claim 3, wherein administering the motor test further comprises the mobile electronic device instructing the patient to follow movement of the dot on the touchscreen.

5. The method of claim 1, further comprising the mobile electronic device presenting to the patient a registration screen.

6. The method of claim 5, wherein presenting the registration screen comprises the mobile electronic device instructing the user to enter a patient identifier and a provider identifier.

7. The method of claim 5, wherein presenting the registration screen comprises the mobile electronic device requesting the user to specify whether to allow the mobile electronic device to record a geographic location of the patient.

8. The method of claim 1, wherein the camera of the mobile electronic device is a front-facing camera.

9. The method of claim 1, wherein the database comprises a remote storage device.

10. The method of claim 1, wherein the mobile electronic device comprises the database.

11. The method of claim 1, further comprising the camera taking at least one of a group consisting of the following: another photo of the at least one eye, another photo of the finger, another photo of the medication.

12. The method of claim 1, wherein the mobile electronic device comprises the database.

13. The method of claim 1, further comprising at least one sensor capturing patient data from the patient.

14. The method of claim 13, further comprising the database storing the patient data captured by the at least one sensor.

15. The method of claim 13, further comprising the mobile electronic device determining whether the at least one sensor is present.

16. The method of claim 13, further comprising sending to a medical provider the photo of the at least one eye, the photo of the finger, the photo of the medication, and the results of the motor test.

17. The method of claim 13, wherein the patient data comprises at least one of a group consisting of the following: heart rate data, blood pressure data, temperature data, Galvanic Skin Response (GSR) data, blood sugar level data, and brain EEG data.

18. A system, comprising:
at least one sensor configured to capture patient data from a patient;
a mobile electronic device including a camera configured to capture a plurality of photos corresponding to the patient, wherein the mobile electronic device is further configured to administer a motor test to the patient; and
a database configured to store the captured patient data, the plurality of photos corresponding to the patient, and results of the motor test.

19. The system of claim 18, wherein the at least one sensor includes at least one from a group consisting of the following: a heart rate sensor, a blood pressure sensor, a temperature sensor, a Galvanic Skin Response (GSR) sensor, a blood sugar level sensor, and a brain EEG sensor.

20. The system of claim 18, wherein the database is remote from the mobile electronic device.
